(19) United States
(12) Patent Application Publication
(10) Pub. No.: US 2024/0217895 A1
(43) Pub. Date: Jul. 4, 2024

(54) PROCESS AND APPARATUS FOR CONVERTING AQUEOUS ALCOHOL TO ETHYLENE

(71) Applicant: UOP LLC, Des Plaines, IL (US)

(72) Inventor: Stanley Joseph Frey, Palatine, IL (US)

(21) Appl. No.: 18/495,423

(22) Filed: Oct. 26, 2023

Related U.S. Application Data
(60) Provisional application No. 63/477,390, filed on Dec. 28, 2022.

(51) Int. Cl. C07C 1/24

(52) U.S. Cl. CPC C07C 1/24

(57) ABSTRACT

A new process and apparatus for preparing aqueous ethanol for dehydration to ethylene entails distilling the aqueous ethanol from water to provide a vaporous ethanol stream and charging the ethanol in the vapor phase to the ethanol dehydration reactor to produce ethylene. The process and apparatus reduce heating requirements and obviates removal of metals and heavy oxygenates that are present in fermented aqueous ethanol streams.

### FIELD

The field is the conversion of alcohols to olefins. The field may particularly relate to the dehydration of ethanol to produce ethylene and the subsequent conversion of the ethylene to long chain olefins and the hydrogenation of the long chain olefins to produce paraffins.

### BACKGROUND

Oil and gas refiners worldwide are exploring methodologies and routes to reduce the carbon footprint in more sustainable processes. An ethanol to jet fuel process is one of the routes that holds promise to minimize or eliminate net carbon combustion. The end product of this process is jet and diesel fuel produced out of bioethanol. The jet fuel is a sustainable aviation fuel intended to replace jet fuel produced out of conventional sources such as crude oil.

Three main steps are followed in the process to convert ethanol to jet fuel. The first is to dehydrate ethanol to produce ethylene. Next the ethylene is converted to long chain olefins and then the long chain olefins are hydrogenated to generate paraffins.

Production of ethanol by fermentation provides an aqueous ethanol stream which comprises a large proportion of water which is removed to a large extent from the ethanol before the dehydration step. Ethanol and water form an azeotrope at atmospheric pressure that cannot be separated easily beyond 95 wt % alcohol. A two-column azeotrope distillation with the addition of an extractant or azeotrope diluent is necessary to purify the alcohol to contain less than 5 wt % water. The aqueous ethanol stream also includes metal contaminants, such as sodium, zinc, phosphates, copper, and calcium and heavy oxygenates also known as fusel oil. The metal contaminants are typically removed in a pretreatment step by adsorption. The heavy oxygenates are typically removed by distillation in a heavy oxygenate removal column upstream of the reaction heaters.

An improved process for removing water would be desirable to prepare the ethanol for dehydrating of ethanol to ethylene.

### SUMMARY

Discovered is a new process and apparatus for preparing aqueous ethanol for dehydration. The process and apparatus envision distilling the aqueous ethanol from water to provide a vaporous ethanol stream and charging the ethanol in the vapor phase to the ethanol dehydration reactor to produce ethylene. The process and apparatus reduce heating requirements and obviate removal of metals and heavy oxygenates that are present in fermented aqueous ethanol streams.

### BRIEF DESCRIPTION OF THE DRAWINGS

FIG. 1 is a schematic process flow diagram of the present disclosure.

### DEFINITIONS

The term “communication” means that material flow is operatively permitted between enumerated components.

The term “downstream communication” means that at least a portion of material flowing to the subject in downstream communication may operatively flow from the object with which it communicates.

The term “upstream communication” means that at least a portion of the material flowing from the subject in upstream communication may operatively flow to the object with which it communicates.

The term “direct communication” means that flow from the upstream component enters the downstream component without passing through a fractionation or conversion unit to undergo a compositional change due to physical fractionation or chemical conversion.

The term “indirect communication” means that flow from the upstream component enters the downstream component after passing through a fractionation or conversion unit to undergo a compositional change due to physical fractionation or chemical conversion.

The term “bypass” means that the object is out of downstream communication with a bypassing subject at least to the extent of bypassing.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take main product from the bottom.

As used herein, the term “a component-rich stream” means that the rich stream coming out of a vessel has a greater concentration of the component than the feed to the vessel.

As used herein, the term “a component-lean stream” means that the lean stream coming out of a vessel has a smaller concentration of the component than the feed to the vessel.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

As used herein, the term “predominant” or “predominate” means greater than 50%, suitably greater than 75% and preferably greater than 90%.

As used herein, the term “Cₓ” are to be understood to refer to molecules having the number of carbon atoms represented by the subscript “x”. Similarly, the term “Cₓ−” refers to molecules that contain less than or equal to x and preferably x and less carbon atoms. The term “Cₓ+” refers to molecules with more than or equal to x and preferably x and more carbon atoms.

As used herein, the term “carbon number” refers to the number of carbon atoms per hydrocarbon molecule and typically a paraffin molecule.

### DETAILED DESCRIPTION

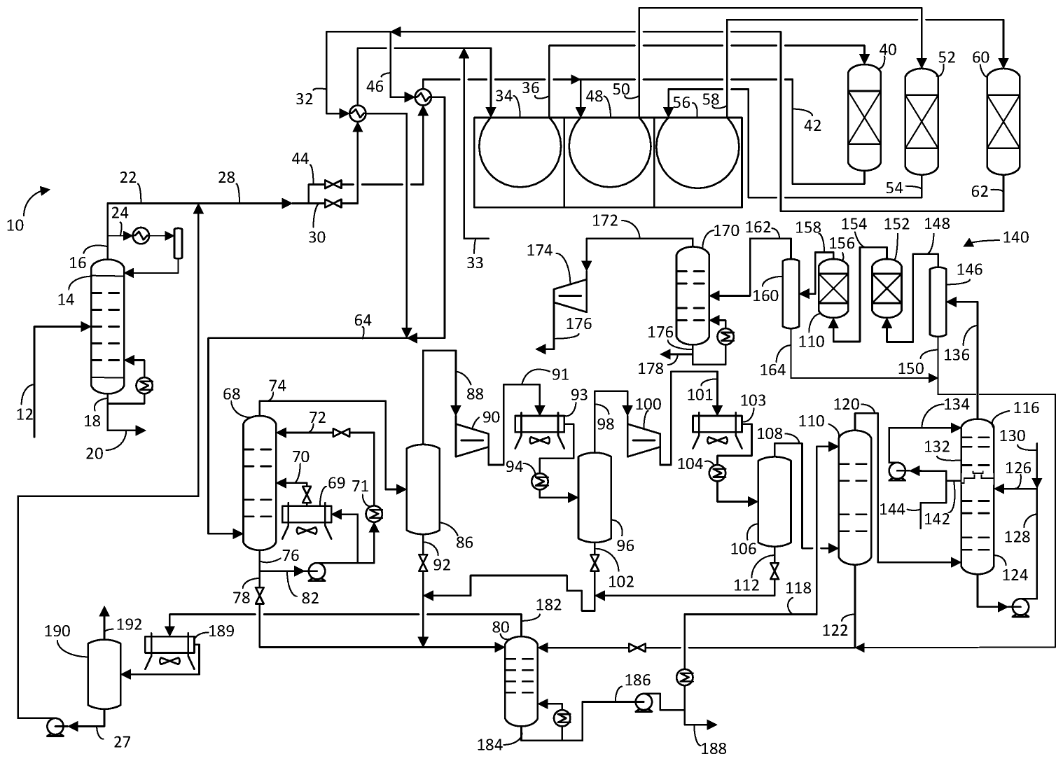

In FIG. 1, in accordance with an exemplary embodiment, a process and apparatus 10 are shown for dehydrating an oxygenate feedstock. The oxygenate feedstock may comprise alcohol and preferably comprises ethanol. The oxygenate feedstock typically is produced from a fermentation process that also produces a large proportion of water. For example, the fermented alcohol stream may comprise over 30 wt % water.

Because the dehydration reaction produces water in large proportion, removal of water from the reactive oxygenate stream to very low concentration is not essential to prepare the alcohol for the catalytic dehydration reaction. Hence, it is proposed to distill the oxygenate stream to provide an overhead gaseous alcohol stream comprising at least 5 wt % water and charging the gaseous alcohol stream to the alcohol dehydration reactor. The energy contained in the vapor stream is much higher than in a condensed liquid stream, so it is proposed to retain the heat in the gaseous alcohol stream to reduce heater duty required to vaporize the stream in route to dehydration reaction temperature.

A feed line 12 transports an oxygenate stream of aqueous ethanol to the process and apparatus 10. The aqueous ethanol stream may comprise a predominance of ethanol and is aqueous comprising at least 30 wt % water. Preferably, the aqueous ethanol is a biorenewable feedstock and/or produced from an alcohol fermentation process.

The aqueous ethanol stream in the feed line 12 typically comprises metals and heavy oxygenates. Heavy oxygenates are also known as fusel oil and include cyclohexanol, cyclopentanol, and heavier acids. Heavy oxygenates comprise at least five carbon atoms. Metal contaminants include sodium, zinc, phosphates, copper, and calcium. The metals are conventionally removed in an adsorbent bed for metals removal, and the heavy oxygenates are conventionally removed in a heavy oxygenates removal column.

The metal and heavy oxygenate laden aqueous ethanol stream in feed line 12 is fed to an ethanol-water azeotropic distillation column 14. The distillation column 14 fractionates a gaseous ethanol stream in an overhead line 16 extending from an overhead of the column and a liquid water stream in a bottoms line 18 extending from a bottom of the column. A reboil stream may be taken from the water stream in the bottoms line 18, reboiled and returned to the column to provide heating requirements to boil the ethanol from the water. A net water stream is taken in a net bottoms line 20 from the water stream in the bottoms line 18. The net water stream comprises all of the metals and the heavy oxygenates from the aqueous ethanol stream in the feed line 12 produced in the distillation column 14. The net water stream comprises at least 99 wt % of the heavy oxygenates and at least 99 wt % metals in the aqueous ethanol stream in the feed line 12.

The gaseous ethanol stream in the overhead line 16 may be comprise at least 5 wt % water, suitably at least 7 wt % water, preferably at least 10 wt % water. However, the gaseous ethanol stream may comprise no more than 21 wt % water. The gaseous ethanol stream may have no more than 1 wt % of the heavy oxygenates or metals from the aqueous ethanol stream. Typically, the gaseous ethanol stream will have no more than 1 ppm of heavy oxygenates or metals. Consequently, the gaseous ethanol stream will not need to be treated in a feed pretreatment section to adsorb metal contaminants or fractionated in a heavy oxygenates removal column to remove heavy oxygenates.

A net gaseous ethanol stream in a net overhead line 22 may be taken from the gaseous ethanol stream in the overhead line 16 while an overhead condenser stream is taken in an overhead condenser line 24 from the gaseous ethanol stream in the overhead line 16. The overhead condenser stream in line 24 is fully condensed and received in a receiver before it is refluxed back to the alcohol-water azeotrope column 14. No third component is added to the receiver to break up the azeotrope between alcohol and water because greater alcohol purity is not necessary for the ethanol dehydration reaction.

The alcohol-water azeotrope column 14 may be operated at around atmospheric pressure with an overhead temperature of about 60° C. (140° F.) to about 90° C. (194° F.) and a bottoms temperature of about 90° C.(194° F.) to about 110° C. (230° F.). Alternatively, the column 14 may be run at elevated pressure not below and preferably above dehydration reaction pressure of about 420 kPa (gauge) 60 psig to about 700 kPa (gauge) (100 psig). Preferably, the alcohol-water azeotrope column 14 is operated at about 350 kPa (50 psid) to about 560 kPa (80 psid) above dehydration pressure to ensure the gaseous ethanol stream can overcome equipment pressure drop to flow through the dehydration reactor 34. In such a case of operating the column 14 at or above dehydration pressure, the overhead temperature would be about 120° C. (248° F.) to about 180° C. (356° F.) and a bottoms temperature of about 150° C. (302° F.) to about 210° C.(410° F.). If the alcohol-water azeotrope column 14 is run at atmospheric pressure or less than reaction pressure, a compressor will be required on the net overhead line 22 to pressure the net gaseous ethanol stream up to reaction pressure.

An ethanol recycle stream in line 27 may be combined with the net gaseous alcohol stream in line 22 to provide a charge gaseous ethanol stream in line 28. The charge gaseous ethanol stream in line 28 is split into two charge gaseous ethanol streams. A first charge gaseous ethanol stream in line 30 is heat exchanged with a first dehydrated exchange stream in line 32, mixed with steam in line 33 and fed to a first charge heater 34. The first charge heater 34 may be a fired heater and may heat the first charge gaseous ethanol stream to about 400° C. to about 550° C. Less heater duty is required to heat the first charge gaseous ethanol stream to reaction temperature required because the gaseous ethanol does not require the enthalpy of vaporization to enter into the vapor phase required for the dehydration reaction. A resulting first heated charge gaseous ethanol stream in line 36 is charged to a first dehydration reactor 40. In the first dehydration reactor 40, gaseous ethanol is converted to ethylene and water over a dehydration catalyst at a pressure of about 455 kPa (gauge) 65 psig to about 630 kPa (gauge) (90 psig). A first dehydrated stream is discharged from the first dehydration reactor 40 in line 42.

A second charge gaseous ethanol stream in line 44 is heat exchanged with a second dehydrated exchange stream in line 46, mixed with the first dehydrated stream in line 42 and fed to a second charge heater 48. The second charge heater 48 may be a fired heater and may heat the second charge stream to about 400° C. to about 550° C. Less heater duty is required to heat the second charge gaseous ethanol stream to reaction temperature required because the gaseous ethanol does not require the enthalpy of vaporization to enter into the vapor phase required for the dehydration reaction. A resulting second heated charge stream in line 50 is charged to a second dehydration reactor 52. In the second dehydration reactor 52, ethanol feed is converted to ethylene and water over a dehydration catalyst at a pressure of about 420 kPa (gauge) 60 psig to about 700 kPa (gauge) (100 psig). A second dehydrated stream is discharged from the second dehydration reactor 52 in line 54.

The second dehydrated stream in line 54 is fed to an interheater 56. The interheater 56 may be a fired heater and may heat the second dehydrated stream to about 400° C. to about 550° C. A resulting third heated charge stream in line 58 is charged to a third dehydration reactor 60. In the third dehydration reactor 60, residual gaseous ethanol feed is converted to ethylene and water over a dehydration catalyst at a pressure of about 420 kPa (gauge) 60 psig to about 700 kPa (gauge) (100 psig). A third dehydrated stream is discharged from the third dehydration reactor 60 in line 62.

The dehydration catalyst is an alumina-based catalyst.

The third dehydrated stream is split between the first dehydrated exchange stream in line 32 and the second dehydrated exchange stream in line 46. The first dehydrated exchange stream in line 32 is heat exchanged with the first charge gaseous ethanol stream in line 30, and the second dehydrated exchange stream in line 46 is heat exchanged with the second charge gaseous ethanol stream in line 44 and the cooled dehydrated streams are recombined in line 64.

The recombined, cooled dehydrated stream in line 64 is fed to a quench tower 68 in which the cooled dehydrated stream is quenched by direct contact with water from a first cooled water stream in line 70 and a second cooled water stream in line 72. A quenched ethylene stream exits in a quench overhead line 74 and a bottoms water stream exits the tower bottoms in line 76. The bottoms water stream is split between a drain stream in line 78 which may be transported to a waste-water stripper column 80 through a valve thereon and a quench recycle stream in line 82. A first portion of the quench recycle stream is air cooled in a product condenser 69 and recycled as the first, lower cooled water stream in line 70 through a valve thereon, and a second portion of the quench recycle stream is heat exchanged in a trim condenser 71 and recycled to the quench tower 68 as the second, higher cooled water stream in line 72. The quench tower 68 may be operated with a bottoms temperature of about 37° C. (100° F.) to about 104° C. (220° F.) and a pressure of about 280 kPa (gauge) (40 psig) to about 490 kPa (gauge) (70 psig) in the overhead.

The quenched ethylene stream in line 74 is fed to a first stage suction drum 86. In the first stage suction drum ethylene exits in the overhead line 88 to a first stage compressor 90 while residual water exits the bottom of the drum in line 92 through a control valve thereon and is transported to the waste-water stripper column 80 perhaps via line 78. The first stage compressor 90 compresses the ethylene stream to a first pressure of about 350 kPa (gauge) (50 psig) to about 1225 kPa (gauge) (175 psig) and the discharge in line 91 is cooled in a first stage discharge cooler 93 and a first stage trim cooler 94.

The cooled, compressed ethylene stream from the first stage trim cooler 94 is fed to a first stage discharge drum 96. From the first stage discharge drum 96 ethylene exits in an overhead line 98 to a second stage compressor 100 while residual water exits a bottom of the drum in line 102 through a control valve thereon and is transported to the waste water stripper column 80 perhaps via lines 92 and 78. The second stage compressor compresses the ethylene stream to a second pressure of about 455 kPa (gauge) (165 psig) to about 3220 kPa (gauge) (460 psig) and the discharge in line 101 is cooled in a second stage discharge cooler 103 and a second stage trim cooler 104.

The twice cooled, compressed ethylene stream from the second stage trim cooler 104 is fed to a second stage discharge drum 106. From the second stage discharge drum 106 ethylene exits in an overhead line 108 and is transported to a water wash tower 110 while a residual water stream exits the bottom of the drum in line 112 through a control valve thereon and is transported to the waste-water stripper column 80 perhaps via lines 102, 92 and 78.

In the water wash tower 110, the twice cooled, compressed ethylene stream is counter-currently washed with cooled, treated water in line 118 from the waste-water stripper column 80 to absorb additional oxygenates to produce a washed ethylene stream exiting in an overhead line 120 and a wash water stream in a bottoms line 122. The washed ethylene stream in the overhead line 120 is transported to a caustic scrubber column 116. The wash water stream in line 122 is transported back to the waste-water stripper column 80 through a valve thereon. The water wash tower 110 may be operated with a bottoms temperature of about 16° C. (60° F.) to about 82° C. (150° F.) and a pressure of about 2800 kPa (gauge) (400 psig) to about 3500 kPa (gauge) (500 psig) in the overhead.

The caustic scrubber column 116 has a lower caustic wash section 124 and an upper water wash section 132. In the lower caustic wash section 124 the washed ethylene stream in line 120 is scrubbed with an aqueous caustic stream from line 126 to absorb acid gases such as carbon dioxide from the washed ethylene stream. Spent caustic is pumped around from the bottom of the lower section in line 128 and replenished with fresh caustic in line 130 to provide the aqueous caustic stream 126. A scrubbed vaporous ethylene stream depleted of acid gases ascends from the caustic wash section 124 to the upper water wash section 132 through a vapor inlet. In the water wash section 132, the scrubbed ethylene stream is contacted with a wash water stream from line 134. A washed, scrubbed vaporous ethylene stream exits the overhead of the water wash section 132 in line 136 and is fed to the product drier section 140. A spent water stream is taken from the bottom of the water wash section 132 from a liquid sump in line 142 and replenished with a fresh water stream from line 144 to provide the wash water stream in line 134 and pumped to the top of the water wash section 132 to be contacted with the scrubbed vaporous ethylene stream. The caustic scrubber column may be operated with a bottoms temperature of about 38° C. (100° F.) to about 43° C. (110° F.) and a pressure of about 2800 kPa (gauge) (400 psig) to about 2975 kPa (gauge) (425 psig) in the overhead.

In the product drier section 140, the washed, scrubbed ethylene stream in line 136 is fed to a first drier inlet knock-out drum 146 to remove residual water and provide a drier inlet stream in line 148 and a knock-out water stream in the bottoms line 150 which is fed to the waste-water stripper column 80 perhaps via line 122. The drier inlet stream is fed to a first product drier 152 in line 148. The first product drier 152 comprises an adsorbent for adsorbing the water from ethylene in the drier inlet stream in line 148 to provide a dried ethylene stream. The adsorbent may be a molecular sieve material with pore diameters of 2-4 A. The first product drier 152 may operate in upflow mode. The product drier section 140 may include a second product drier 156 that operates as the first product drier 142. The two product driers may be operated in series but are preferably arranged in a lead-lag operation to facilitate regeneration during continuous operation. The second product drier 156 comprises an adsorbent for adsorbing the water from ethylene like in the first product drier 152. A dried ethylene stream exits the product drier section 140 in a dried ethylene stream in line 158. The product drier section 140 may be operated at a temperature of about 32° C. (90° F.) to about 49° C. (120° F.) and a pressure of about 2.8 MPa (gauge) (400 psig) to about 3.1 MPa (gauge) 450 psig).

The dried ethylene stream in line 158 is fed to a drier outlet knock-out drum 160 to remove residual water and provide a drier outlet stream in line 162 and a second knock-out water stream in a bottoms line 164 which is fed to the waste-water stripper column 80 perhaps via lines 150 and 122.

The drier outlet stream in line 162 may be fed to a heavy oxygenates removal column 170 to separate an overhead stream comprising predominantly ethylene but perhaps higher olefins from heavy ketones and diethyl ether. The olefins are produced in an overhead line 172 and fed to a third stage compressor 174 and a bottoms heavy oxygenate stream is produced in a bottoms line 176. A heavy oxygenate purge stream may be taken in line 178 to heavy oxygenate treatment while a reboil portion is reboiled and fed back to the column 170. A compressed ethylene stream at a pressure of about 2800 kPa (gauge) (400 psig) to about 7000 kPa (gauge) (1000 psig) in a compressor discharge line 176 may be provided to a dimerization section. The heavy oxygenate removal column 170 may be operated with a bottoms temperature of about −29° C. (−20° F.) to about 121° C. (250° F.) and a pressure of about 2.4 MPa (gauge) (350 psig) to about 3.1 MPa (gauge) (450 psig) in the overhead.

Water streams comprising oxygenates and volatiles in lines 92, 102, 112, 122, 150, 164 may be fed to the waste-water stripper column 80 in which volatiles and oxygenates are boiled off to provide an overhead volatile stream in line 182 and a stripped water stream in line 184. A portion of the stripped water stream can be reboiled and fed back to the column to provide necessary heat. A treated water stream in line 186 may be pumped to water outlets which includes other water outlets in line 188 and the cooled, treated water stream in line 118 to the water wash tower 110. The waste-water stripper column 80 may be operated with a bottoms temperature of about 93° C. (200° F.) to about 121° C. (250° F.) and a pressure of about 35 kPa (gauge) (5 psig) to about 138 kPa (gauge) (20 psig) in the overhead.

The overhead volatile stream in line 182 may be cooled in an air cooler 189 and fed to an off-gas knock out drum 190. An overhead stream from the knock-out drum 190 in line 192 may be sent to flare while an ethanol recycle stream is pumped to the feed surge drum 26 in line 27 perhaps via line 24.

The process and apparatus disclosed conserve heat in the gaseous ethanol stream to avoid having to re-vaporize the charge ethanol stream after removing the bulk of water from the aqueous ethanol stream in the alcohol-water azeotrope column 14. Moreover, by maintaining the overhead ethanol stream in the vapor phase metals and heavy oxygenates are prevented from entering the gaseous overhead ethanol stream obviating a metals pretreatment adsorption unit and a heavy oxygenate removal column.

### EXAMPLE

We simulated operating the ethanol-water azeotropic distillation column at 10 atmospheres instead of the typical 1 atmosphere of pressure. The higher pressure would enable transport of the overhead gaseous ethanol stream to the ethanol dehydration reactor without a compressor. We found the water concentration in the overhead gaseous ethanol stream only increased by 5 to 10 wt % at the much higher pressure. We were surprised to find the additional water concentration in the gaseous ethanol stream was too low to affect the ethanol dehydration reaction which is well within the water tolerance due to the large presence of water in the dehydration reaction.

### SPECIFIC EMBODIMENTS

While the following is described in conjunction with specific embodiments, it will be understood that this description is intended to illustrate and not limit the scope of the preceding description and the appended claims.

A first embodiment of the disclosure is a process for producing ethylene comprising feeding an aqueous ethanol steam to a distillation column to produce a gaseous ethanol stream in an overhead of the distillation column and a liquid water stream in the bottom of the distillation column; and charging the gaseous ethanol stream to a dehydration reactor comprising dehydration catalyst to convert ethanol to ethylene. An embodiment of the disclosure is one, any or all of prior embodiments in this paragraph up through the first embodiment in this paragraph wherein the aqueous ethanol stream comprises at least 30 wt % water. An embodiment of the disclosure is one, any or all of prior embodiments in this paragraph up through the first embodiment in this paragraph wherein the gaseous ethanol stream comprises no more than 21 wt % water. An embodiment of the disclosure is one, any or all of prior embodiments in this paragraph up through the first embodiment in this paragraph wherein the liquid water stream comprises at least 99 wt % of the heavy oxygenates and metals. An embodiment of the disclosure is one, any or all of prior embodiments in this paragraph up through the first embodiment in this paragraph wherein the gaseous ethanol stream comprises at least 5 wt % water. An embodiment of the disclosure is one, any or all of prior embodiments in this paragraph up through the first embodiment in this paragraph further comprising charging the gaseous ethanol stream to the dehydration reactor in the vapor phase. An embodiment of the disclosure is one, any or all of prior embodiments in this paragraph up through the first embodiment in this paragraph wherein the gaseous ethanol stream is compressed up to dehydration reaction pressure. An embodiment of the disclosure is one, any or all of prior embodiments in this paragraph up through the first embodiment in this paragraph further comprising operating the distillation column at no less than dehydration reaction pressure. An embodiment of the disclosure is one, any or all of prior embodiments in this paragraph up through the first embodiment in this paragraph wherein the gaseous ethanol stream comprises no more than 1 wt % of heavy oxygenates from the aqueous ethanol stream. An embodiment of the disclosure is one, any or all of prior embodiments in this paragraph up through the first embodiment in this paragraph wherein the gaseous ethanol stream comprises no more than 1 wt % of metals from the aqueous ethanol stream. An embodiment of the disclosure is one, any or all of prior embodiments in this paragraph up through the first embodiment in this paragraph further comprising heating the gaseous ethanol stream to dehydration temperature.

A second embodiment of the disclosure is an apparatus for producing an ethanol stream comprising an alcohol-water distillation column; a heater in direct downstream communication with the alcohol-water distillation column; and a catalytic dehydration reactor in direct downstream communication with the heater. An embodiment of the disclosure is one, any or all of prior embodiments in this paragraph up through the second embodiment in this paragraph wherein the heater is in direct downstream communication with the alcohol-water distillation column.

A third embodiment of the disclosure is a process for producing ethylene comprising feeding an aqueous ethanol steam to a distillation column to produce a gaseous ethanol stream in an overhead of the distillation column and a liquid water stream in the bottom of the distillation column; and charging the gaseous ethanol stream comprising at least 30 wt % water to a dehydration reactor comprising dehydration catalyst to convert ethanol to ethylene. An embodiment of the disclosure is one, any or all of prior embodiments in this paragraph up through the third embodiment in this paragraph wherein the gaseous ethanol stream comprises no more than 21 wt % water. An embodiment of the disclosure is one, any or all of prior embodiments in this paragraph up through the third embodiment in this paragraph wherein the liquid water stream comprises at least 99 wt% of the heavy oxygenates and metals. An embodiment of the disclosure is one, any or all of prior embodiments in this paragraph up through the third embodiment in this paragraph wherein the gaseous ethanol stream comprises at least 5 wt % water. An embodiment of the disclosure is one, any or all of prior embodiments in this paragraph up through the third embodiment in this paragraph further comprising charging the gaseous ethanol stream to the dehydration reactor in the vapor phase. An embodiment of the disclosure is one, any or all of prior embodiments in this paragraph up through the third embodiment in this paragraph wherein the gaseous ethanol stream is compressed up to dehydration reaction pressure. An embodiment of the disclosure is one, any or all of prior embodiments in this paragraph up through the third embodiment in this paragraph further comprising operating the distillation column at no less than dehydration reaction pressure.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for producing ethylene comprising:
feeding an aqueous ethanol steam to a distillation column to produce a gaseous ethanol stream in an overhead of the distillation column and a liquid water stream in the bottom of the distillation column; and
charging said gaseous ethanol stream to a dehydration reactor comprising dehydration catalyst to convert ethanol to ethylene.

2. The process of claim 1 wherein said aqueous ethanol stream comprises at least 30 wt % water.

3. The process of claim 1 wherein said gaseous ethanol stream comprises no more than 21 wt % water.

4. The process of claim 1 wherein said liquid water stream comprises at least 99 wt % of the heavy oxygenates and metals.

5. The process of claim 1 wherein said gaseous ethanol stream comprises at least 5 wt % water.

6. The process of claim 1 further comprising charging said gaseous ethanol stream to the dehydration reactor in the vapor phase.

7. The process of claim 1 wherein said gaseous ethanol stream is compressed up to dehydration reaction pressure.

8. The process of claim 1 further comprising operating the distillation column at no less than dehydration reaction pressure.

9. The process of claim 1 wherein the gaseous ethanol stream comprises no more than 1 wt % of heavy oxygenates from the aqueous ethanol stream.

10. The process of claim 1 wherein the gaseous ethanol stream comprises no more than 1 wt % of metals from the aqueous ethanol stream.

11. The process of claim 1 further comprising heating the gaseous ethanol stream to dehydration temperature.

12. An apparatus for producing an ethanol stream comprising:
an alcohol-water distillation column;
a heater in direct downstream communication with said alcohol-water distillation column; and
a catalytic dehydration reactor in direct downstream communication with said heater.

13. The apparatus of claim 11 wherein said heater is in direct downstream communication with said alcohol-water distillation column.

14. A process for producing ethylene comprising:
feeding an aqueous ethanol steam to a distillation column to produce a gaseous ethanol stream in an overhead of the distillation column and a liquid water stream in the bottom of the distillation column; and
charging said gaseous ethanol stream comprising at least 30 wt % water to a dehydration reactor comprising dehydration catalyst to convert ethanol to ethylene.

15. The process of claim 1 wherein said gaseous ethanol stream comprises no more than 21 wt % water.

16. The process of claim 1 wherein said liquid water stream comprises at least 99 wt % of the heavy oxygenates and metals.

17. The process of claim 1 wherein said gaseous ethanol stream comprises at least 5 wt % water.

18. The process of claim 1 further comprising charging said gaseous ethanol stream to the dehydration reactor in the vapor phase.

19. The process of claim 1 wherein said gaseous ethanol stream is compressed up to dehydration reaction pressure.

20. The process of claim 1 further comprising operating the distillation column at no less than dehydration reaction pressure.
